(19) United States
(12) Patent Application Publication
(10) Pub. No.: US 2004/0234480 A1
(43) Pub. Date: Nov. 25, 2004

(54) Use of oligomeric proanthrocyanidins

(76) Inventors: Gilles Pauly, Nancy (FR); Florence Henry, Villers-les-Nancy (FR); Louis Danoux, Saulxures les Nancy (FR)

Correspondence Address: COGNIS CORPORATION, PATENT DEPARTMENT, 300 BROOKSIDE AVENUE, AMBLER, PA, 19002, US

(21) Appl. No.: 10/477,081

(22) PCT Filed: May 3, 2002
(86) PCT No.: PCT/EP02/04861

(30) Foreign Application Priority Data
May 10, 2001 (EP) 014011902

(51) Int. Cl. A61K 7/06; A61K 7/11; A61K 35/78

(52) U.S. Cl. 424/70.13; 514/27; 514/456; 424/74; 424/729; 424/766; 424/770

(57) ABSTRACT

Methods of treating skin to counteract the effects of ageing as well as conditions such as acne, rosacea and cellulite along with methods of counteracting specific adverse biochemical reactions in the dermal region are disclosed, wherein the methods comprise (a) providing a skin substrate to be treated; and contacting the skin substrate with a cosmetic preparation comprising at least one oligomeric proanthocyanidin.

### FIELD OF THE INVENTION

The invention is in the field of cosmetics and relates to the use of oligomeric proanthocyanidins for the preparation of skin treatment products, specifically products which counteract skin ageing.

### PRIOR ART

Presumably it is not only since Cleopatra's time, who is known to have been accustomed to taking baths in ass's milk, that there has been a desire to preserve the freshness and beauty of youthful skin into old age. If the approaches in the past were limited more to the surface of the skin and in many cases were purely empirical, the last two decades have produced a lot of knowledge about the influence of environmental factors and their intervention in the metabolism. This understanding of the mechanisms is nowadays—and probably to a greater degree in the future—used to pursue new ways of rigorously counteracting skin ageing in order—who knows?—one day to perhaps finally achieve Cleopatra's goal.

However, there is still some way to go to reach this point and it is a question of tackling the environmental factors which form the essential hindrances to this. It is of course known that UV-A rays produce reactive oxygen free radicals (reactive oxygen species=ROS) in human cells and are therefore presumably responsible for the effect of skin ageing. It is also known to the person skilled in the art that certain antioxidants which occur in the natural metabolism, such as, for example, flavins, porphyrins, pterins, but also NADH and NADPH, fully react with these ROS and can thus counteract the harmful effects of the free radicals to a certain extent [Cosmètologie, 11, 32-36 (1996)].

As well as this primary effect, there is also a secondary effect which UV rays advantageously trigger in older cells. For example, Gedaleta et al., reported in Biochimie, 80(10), 863-870 (1998) on an ROS-induced increase in the activity in the mitochondria, which leads to an excessive output of matrix-metallo proteinases (MMP), in particular of zinc-containing collagenases, gelatinases and stromelysin. However, MMPs have the property of attacking the dermal macromolecules of the connective tissue, such as proteoglycan, collagen and elastin, of breaking peptide bonds and thus likewise being a contributory cause of skin ageing. During inflammatory processes in the skin as well, proteases, such as, for example, the serine protease elastase or matrix-metallo proteases (MMP), such as collagenase, and a further elastin-degrading elastase, which is a type of MMP, are released by the macrophages and the polymorphonuclear neutrophilic granulocytes.

A common method of counteracting these effects consists in increasing the concentration of natural MMP inhibitors which are present anyway in the metabolism, or in adding synthetic MMP inhibitors. Examples thereof are, for example, cysteine, TIMP (tissue inhibitor of matrix proteinase), o-phenanthroline or hydroxamic acid. The investigations by Fisher at al. [J. Clin. Invest. 101, 1432-1440 (1998); Nature 379, 335-339 (1996)] investigate deeper into the mechanism of the MMPs and propose the use of such substances which counteract the UV-B-controlled activation of the AP-1. Thus, for example, it was shown that retinoic acid and dexamethasone suppress the AP-1 transcription factor which is responsible for stimulating the MMP gene.

A further group of substances which are named in connection with the inhibition of MMP are the oligomeric proanthocyanidins (OPC) and flavonoids, which were isolated for the first time by Masquellier from Vitis vinifera in 1947. Masquellier also recognized for the first time the property of OPC in being able to scavenge free radicals [U.S. Pat. No. 4,698,360]. In fact, the OPCs do not inhibit the MMPs directly, but protect collagen and elastin fibers against attack by proteinases. Investigations by Robert et al. showed that the proanthrocyanidin A2 from Vitis vinifera is an effective antioxidant both for hydrophilic and lipophilic free radicals, provides moderate elastase inhibition, while the property of inhibiting collagenase is less marked. [J. Med. Esth. Et Chir. 10, 211-217 (1993)].

However, it would then be incorrect to put the phenomenon of skin ageing purely down to MMP activity. In actual fact, there is regrettably a large number of other factors, such as, for example, the influence of hydrogen peroxide, which leads to the degradation of Fe—S clusters in the enzymes of the mitochondrial respiratory chain. The iron ions liberated in the process migrate into the cytoplasm and can liberate hydroxyl radicals, this taking place either in the sense of a Fenton reaction directly from H₂O₂, or as a Haber-Weiss reaction via the detour of the formation of the superoxide anion.

This short overview is intended to illustrate that the effect of skin ageing cannot be dealt with by a “cure-all”, which reliably eliminates a single disruptive parameter. What is sought instead is substances which provide a broad spectrum of effects and thus combat as many causes as possible at the same time. Such a substance should, for example,

have a strong affinity to iron ions and form inactive, acceptable iron complexes;

react fully with superoxide anions to give harmless products;

both inhibit MMP activity and also prevent a resulting increased release of MMP particularly in older dermal fibroblasts;

suppress the respiratory burst of human leucocytes.

The object of the present invention was consequently to provide substances with the described complex profile of requirements.

### DESCRIPTION OF THE INVENTION

The invention provides the use of oligomeric proanthocyanidins (OPC) for the preparation of skin treatment compositions, specifically of anti-ageing products, which counteract skin ageing.

Surprisingly, it has been found that OPC, and specifically the dimeric A2 forms, counteract skin ageing by simultaneously

both inhibiting MMP, and also primarily significantly reducing the activity and the output of MMP, specifically in older fibroblasts;

stimulating the cells to protect themselves against spontaneous ageing processes and oxidative stress which UV rays or free radicals trigger;

forming inactive complexes with iron ions;

deactivating superoxide anions;

preventing the release of proinflammatory substances from the mastocytes, and the basophilic and/or eusinophilic leucocytes;

inhibiting plasmin (human serine protease).

On the basis of these effects—shown in the examples below by experimental results—oligomeric proanthocyanidins can be used in order to

protect sensitive skin;

act against skin disorders, in particular acne and rosacea, and

prevent the relaxation of connective tissue (cellulite).

The invention encompasses the knowledge that both the OPCs and specifically the dimeric A2 forms have these properties, and also enriched products, as can be obtained by extraction from specific fruits, seeds, plants and parts of plants. In some cases, even better results are achieved with these extracts since these still contain bioactive ingredients which are synergistically effective with one another.

### Oligomeric Proanthocyanidins (OPC)

The first oligomeric proanthocyanidins were isolated by Masquellier from grape seeds. As monomer building blocks, they comprise tannins, which are widespread in the plant kingdom. From a chemical point of view, a distinction may be made between two types of tannins, namely condensed forms, to which the procyanidin A2 also belongs, and hydrolyzable tannins. Condensed tannins, which are also referred to as flavan-3-ol, are formed in biosynthesis as a result of condensation of monomers, such as, for example, catechin, gallocatechin and afzelechin (2-R, 3-S type monomers), and epicatechin, epigallocatechin and epiafzelechin (2-R, 3-R type monomers). Condensation of the monomers produces firstly dimers and then higher oligomers, the condensation taking place as a result of the formation of a C-C bond in 4-8 or 6-8 position. In the case of the preferred A2 dimers of the proanthrocyanidin A2 type there is a double bond, namely C2>0>C7 and C4>C8. The structure is shown in the diagram below:

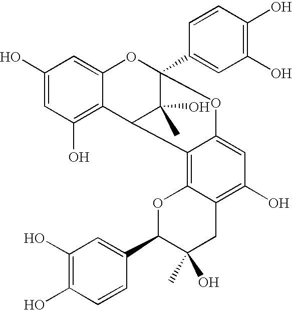

The A2 type proanthocyanidins are less susceptible to hydrolysis than the B types. Moreover, this term is used synonymously for the group of condensed tannins since the latter cleave off monomers under the influence of hot mineral acids. The proanthocyanidins may in principle be synthetic in nature, but from a practical point of view enrichment products with an effective amount of the OPCs of A2 dimers are preferably suitable; these may be obtained by extraction of certain fruits, seeds, plants or parts of plants. Suitable sources are, in particular, green tea, pine bark, grape seed (Vitis vinifera), Litchi pericarp (Litchi chinensis) and Potentilla (Potentilla erecta), and mixtures thereof. The effective use amount is—based on pure OPC—usually in the range from 0.0001 to 1% by weight, preferably 0.001 to 0.5% by weight and in particular 0.01 to 0.1% by weight. Thus, for example, in the case of Litchi pericarp, the effective amount of A2 dimers is at least 0.50 g/100 g, preferably at least 0.60 g/100 g and particularly preferably 0.65 g/100 g.

### Extraction

The extracts can be prepared in a manner known per se, i.e. for example by aqueous, alcoholic or aqueous-alcoholic extraction from the plants or parts of plants. With regard to the suitable conventional extraction methods, such as maceration, remaceration, digestion, agitation maceration, fluidized-bed extraction, ultrasound extraction, countercurrent extraction, percolation, repercolation, evacolation (extraction under reduced pressure), diacolation and solid-liquid extraction under continuous reflux which is carried out in a Soxhlet extractor, all of which are known to the person skilled in the art and can be used in principle, reference may be made, for the sake of simplicity, to, for example, Hagers Handbuch der Pharmazeutischen Praxis (5^{th} Edition, Vol. 2, pp. 1026-1030, Springer Verlag, Berlin-Heidelberg-New York 1991). The percolation method is advantageous for industrial use. Starting materials which may be used are fresh plants or parts of plants, although usually the starting materials are dried plants and/or parts of plants which can be mechanically comminuted prior to extraction. In this connection, all comminution methods known to the person skilled in the art are suitable, freeze-grinding being given by way of example. Solvents which can be used for carrying out the extractions are organic solvents, water (preferably hot water at a temperature greater than 80° C. and in particular greater than 95° C.), or mixtures of organic solvents and water, in particular low molecular weight alcohols with greater or lesser water content. Particular preference is given to extraction with methanol, ethanol, pentane, hexane, heptane, acetone, propylene glycols, polyethylene glycols, and ethyl acetate, and mixtures thereof, and aqueous mixtures thereof. The extraction usually takes place at 20 to 100° C., preferably at 30 to 90° C., in particular at 60 to 80° C. In a preferred embodiment, the extraction is carried out under an inert gas atmosphere to avoid oxidation of the active ingredients of the extract. This is of importance particularly for extractions at temperatures above 40° C. The extraction times are adjusted by the person skilled in the art depending on the starting material, the extraction method, the extraction temperature, the ratio of solvent to raw material, etc. After the extraction, the resulting crude extracts can optionally be subjected to further customary steps, such as, for example, purification, concentration and/or decoloration. If desired, the extracts prepared in this way can, for example, be subjected to selective removal of individual undesired ingredients. The extraction can be carried out to any desired degree of extraction, but is usually carried out exhaustively. Typical yields (=amount of dry substance in the extract based on the amount of raw material used) in the extraction of dried leaves are in the range from 3 to 15% by weight, in particular 6 to 10% by weight. The present invention encompasses the knowledge that the extraction conditions and also the yields of the end extracts can be chosen by the person skilled in the art depending on the desired field of use. These extracts which usually have active substance contents (=solids contents) in the range from 0.5 to 10% by weight can be used as they are, although it is likewise possible to completely remove the solvent by drying, in particular by spray-drying or freeze-drying, in which case an intensively red-colored solid is left behind. The extracts can also be used as starting materials for obtaining the abovementioned pure active ingredients if the latter cannot not be prepared in a simple and cost-effective manner by a synthetic route. Consequently, the active ingredient content in the extracts can be 5 to 100% by weight, preferably 50 to 95% by weight. The extracts themselves can be in the form of aqueous preparations and/or preparations dissolved in organic solvents, and also in the form of spray-dried and freeze-dried, anhydrous solids. Suitable organic solvents in this connection are, for example, the aliphatic alcohols having 1 to 6 carbon atoms (e.g. ethanol), ketones (e.g. acetone), halogenated hydrocarbons (e.g. chloroform or methylene chloride), lower esters or polyols (e.g. glycerol or glycols).

### Skin Treatment Compositions

The skin treatment compositions obtainable with the inventive use of the OPCs may comprise, as further auxiliaries and additives, mild surfactants, oily bodies, emulsifiers, pearlescent waxes, bodying agents, thickeners, super fatty agents, stabilizers, polymers, silicone compounds, fats, waxes, lecithins, phospholipids, biogenic active ingredients, UV light protection factors, antioxidants, deodorants, antiperspirants, film formers, swelling agents, insect repellents, self-tanning agents, tyrosine inhibitors (depigmentation agent), hydrotropes, solubilizers, preservatives, perfume oils, dyes and the like.

### Surfactants

Surface-active substances which may be present are anionic, nonionic, cationic and/or amphoteric or zwitterionic surfactants, the content of which in the compositions is usually about 1 to 70% by weight, preferably 5 to 50% by weight and in particular 10 to 30% by weight. Typical examples of anionic surfactants are soaps, alkylbenzenesulfonates, alkanesulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular wheat-based vegetable products) and alkyl (ether) phosphates. If the anionic surfactants contain polyglycol ether chains, these may have a conventional homolog distribution, but preferably have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. If the nonionic surfactants contain polyglycol ether chains, these may have a conventional homolog distribution, but preferably have a narrowed homolog distribution. Typical examples of cationic surfactants are quaternary ammonium compounds, such as, for example, dimethyldistearylammonium chloride, and ester quats, in particular quaternized fatty acid trialkanolamine ester salts. Typical examples of amphoteric or zwitterionic surfactants are alkylbetaines, alkylamidobetaines, aminopropionates, aminoglycinates, imidazolinium-betaines and sulfobetaines. Said surfactants are exclusively known compounds. With regard to structure and preparation of these substances, reference may be made to relevant review works, for example, J. Falbe (ed.), “Surfactants in Consumer Products”, Springer Verlag, Berlin, 1987, pp. 54-124 or J. Falbe (ed.), “Katalysatoren, Tenside und Mineralöladditive”, Thieme Verlag, Stuttgart, 1978, pp. 123-217. Typical examples of particularly suitable mild, i.e. particularly skin-compatible surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamidobetaines, amphoacetals and/or protein fatty acid condensates, the latter preferably based on wheat proteins.

### Oily Bodies

Suitable oily bodies are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkylhydroxycarboxylic acids with linear or branched C₆-C₂₂-fatty alcohols (cf. DE 19756377 A1), in particular dioctyl malates, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/ di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonates (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or unsymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicon methicone types, inter alia) and/or aliphatic or naphthenic hydrocarbons, such as, for example, such as squalane, squalene or dialkylcyclohexanes.

### Emulsifiers

Suitable emulsifiers are, for example, nonionogenic surfactants from at least one of the following groups:

addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group, and onto alkylamines having 8 to 22 carbon atoms in the alkyl radical;

alkyl and/or alkenyl oligoglycosides having 8 to 22 carbon atoms in the alk(en)yl radical and the ethoxylated analogs thereof;

addition products of from 1 to 15 mol of ethylene oxide to castor oil and/or hydrogenated castor oil;

addition products of from 15 to 60 mol of ethylene oxide to castor oil and/or hydrogenated castor oil;

partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mol of ethylene oxide;

partial esters of polyglycerol (average degree of self-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide;

mixed esters of pentaerythritol, fatty acids, citric acid and fatty alcohol as in German Patent 1165574 and/or mixed esters of fatty acids having 6 to 22 carbon atoms, methylglucose and polyols, preferably glycerol or polyglycerol,

mono-, di- and trialkyl phosphates, and mono-, di- and/or tri-PEG alkyl phosphates and salts thereof;

wool wax alcohols;

polysiloxane-polyalkyl-polyether copolymers and corresponding derivatives;

block copolymers, e.g. polyethylene glycol-30 dipolyhydroxystearates;

polymer emulsifiers, e.g. Pemulen grades (TR-1, TR-2) from Goodrich;

polyalkylene glycols, and

glycerol carbonate.

Ethylene Oxide Addition Products

The addition products of ethylene oxide and/or of propylene oxide onto fatty alcohols, fatty acids, alkylphenols or onto castor oil are known, commercially available products. These are homolog mixtures whose average degree of alkoxylation corresponds to the ratio of the amounts of ethylene oxide and/or propylene oxide and substrate with which the addition reaction is carried out. C_{12/18}-fatty acid mono- and diesters of addition products of ethylene oxide onto glycerol are known from German Patent 2024051 as refatting agents for cosmetic preparations.

Alkyl and/or Alkenyl Oligoglycosides

Alkyl and/or alkenyl oligoglycosides, their preparation and their use are known from the prior art. They are prepared, in particular, by reacting glucose or oligo-saccharides with primary alcohols having 8 to 18 carbon atoms. With regard to the glycoside radical, both monoglycosides, in which a cyclic sugar radical is glycosidically bonded to the fatty alcohol, and also oligomeric glycosides having a degree of oligomerization of up to, preferably, about 8, are suitable. The degree of oligomerization here is a statistical average value which is based on a homolog distribution customary for such technical-grade products.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid moglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride, and the technical-grade mixtures thereof which may also comprise small amounts of triglyceride as a minor product of the preparation process. Likewise suitable are addition products of 1 to 30 mol, preferably 5 to 10 mol, of ethylene oxide to said partial glycerides.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Likewise suitable are addition products of 1 to 30 mol, preferably 5 to 10 mol, of ethylene oxide to said sorbitan esters.

Polyglycerol Esters

Typical examples of suitable polyglycerol esters are polyglyceryl-2 dipolyhydroxystearate (Dehymuls® PGPH), polyglycerol-3 diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate, and mixtures thereof. Examples of further suitable polyol esters are the mono-, di- and triesters, optionally reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Anionic Emulsifiers

Typical anionic emulsifiers are aliphatic fatty acids having 12 to 22 carbon atoms, such as, for example, palmitic acid, stearic acid or behenic acid, and dicarboxylic acids having 12 to 22 carbon atoms, such as, for example, azeleic acid or sebaccic acid.

Amphoteric and Cationic Emulsifiers

Furthermore, zwitterionic surfactants can be used as emulsifiers. The term “zwitterionic surfactants” refers to those surface-active compounds which carry at least one quaternary ammonium group and at least one carboxylate and one sulfonate group in the molecule. Particularly suitable zwitterionic surfactants are the betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethylhydroxyethylcarboxymethyl glycinate. Particular preference is given to the fatty acid amide derivative known under the CTFA name Cocamidopropyl Betaine. Likewise suitable emulsifiers are ampholytic surfactants. The term “ampholytic surfactants” means those surface-active compounds which, apart from a C_{8/18}-alkyl or -acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkyl aminopropionate, cocoacylaminoethyl aminopropionate and C_{12/18}-acylsarcosine. Finally, cationic surfactants are also suitable emulsifiers, those of the ester quat type, preferably methyl-quaternized difatty acid triethanolamine ester salts, being particularly preferred.

### Fats and Waxes

Typical examples of fats are glycerides, i.e. solid or liquid vegetable or animal products which consist essentially of mixed glycerol esters of higher fatty acids, suitable waxes are inter alia natural waxes, such as, for example, candelilla wax, carnauba wax, Japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microcrystalline waxes; chemically modified waxes (hard waxes), such as, for example, montan ester waxes, sasol waxes, hydrogenated jojoba waxes, and synthetic waxes, such as, for example, polyalkylene waxes and polyethylene glycol waxes. In addition to the fats, suitable additives are also fat-like substances, such as lecithins and phospholipids. The term lecithins is understood by the person skilled in the art as meaning those glycerophospholipids which form from fatty acids, glycerol, phosphoric acid and choline by esterification. Lecithins are thus frequently also referred to as phosphatidylcholines (PC). Examples of natural lecithins which may be mentioned are the cephalins, which are also referred to as phosphatidic acids and represent derivatives of 1,2-diacyl-sn-glycerol-3-phosphoric acids. By contrast, phospholipids are usually understood as meaning mono- and, preferably, diesters of phosphoric acid with glycerol (glycerophosphates), which are generally considered to be fats. In addition, sphingosines and sphingolipids are also suitable.

### Pearlescent Waxes

Examples of suitable pearlescent waxes are: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

### Bodying Agents and Thickeners

Suitable bodying agents are primarily fatty alcohols or hydroxy fatty alcohols having 12 to 22, and preferably 16 to 18, carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, Aerosil grades (hydrophilic silicas), polysaccharides, in particular xanthan gum, guar guar, agar agar, alginates and Tyloses, carboxymethylcellulose and hydroxyethyl- and hydroxypropylcellulose, and also relatively high molecular weight polyethylene glycol mono- and diesters of fatty acids, polyacrylates (e.g. Carbopols® and Pemulen grades from Goodrich; Synthalens® from Sigma; Keltrol grades from Kelco; Sepigel grades from Seppic; Salcare grades from Allied Colloids), polyacrylamides, polymers, polyvinyl alcohol and polyvinylpyrrolidone. Bentonites have proved to be particularly effective, such as, for example, Bentone® Gel VS-5PC (Rheox), which is a mixture of cyclopentosiloxane, disteardimonium hectointe and propylene carbonate. Also suitable are surfactants, such as, for example, ethoxylated fatty acid glycerides, esters of fatty acids with polyols such as, for example, pentaerythritol or trimethylolpropane, fatty alcohol ethoxylates having a narrowed homolog distribution or alkyl oligoglucosides, and electrolytes such as sodium chloride and ammonium chloride.

### Superfatting Agents

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter also serving as foam stabilizers.

### Stabilizers

Stabilizers which can be used are metal salts of fatty acids, such as, for example, magnesium, aluminum and/or zinc stearate or zinc ricinoleate.

### Polymers

Suitable cationic polymers are, for example, cationic cellulose derivatives, such as, for example, a quaternized hydroxyethylcellulose obtainable under the name Polymer JR 400® from Amerchol, cationic starch, copolymers of diallylammonium salts and acrylamides, quaternized vinylpyrrolidone-vinylimidazole polymers, such as, for example, Luviquat® (BASF), condensation products of polyglycols and amines, quaternized collagen polypeptides, such as, for example, lauryldimonium hydroxypropyl hydrolyzed collagen (Lamequat® L/Griünau), quaternized wheat polypeptides, polyethyleneimine, cationic silicone polymers, such as, for example, amodimethicones, copolymers of adipic acid and dimethylaminohydroxypropyldiethylenetriamine (Cartaretins®/Sandoz), copolymers of acrylic acid with dimethyldiallylammonium chloride (Merquat® 550/Chemviron), polyaminopolyamides, as described, for example, in FR 2252840 A, and crosslinked water-soluble polymers thereof, cationic chitin derivatives, such as, for example, quaternized chitosan, optionally in micro-crystalline dispersion, condensation products from dihaloalkyls, such as, for example, dibromobutane with bisdialkylamines, such as, for example, bis-dimethyl-amino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol® AZ-1 from Miranol.

Suitable anionic, zwitterionic, amphoteric and nonionic polymers are, for example, vinyl acetate-crotonic acid copolymers, vinylpyrrolidone-vinyl acrylate copolymers, vinyl acetate-butyl maleate-isobornyl acrylate copolymers, methyl vinyl ether-maleic anhydride copolymers and esters thereof, uncrosslinked polyacrylic acids and polyacrylic acids crosslinked with polyols, acrylamidopropyltrimethylammonium chloride-acrylate copolymers, octylacrylamide-methyl methacrylate-tert-butylaminoethyl methacrylate-2-hydroxypropyl methacrylate copolymers, polyvinylpyrrolidone, vinylpyrrolidone-vinyl acetate copolymers, vinylpyrrolidone-dimethylaminoethyl methacrylate-vinylcaprolactam terpolymers, and optionally derivatized cellulose ethers and silicones. Further suitable polymers and thickeners are listed in Cosm. Toil. 108, 95 (1993).

### Silicone Compounds

Suitable silicone compounds are, for example, dimethylpolysiloxanes, methylphenylpolysiloxanes, cyclic silicones, and amino-, fatty-acid-, alcohol-, polyether-, epoxy-, fluorine-, glycoside- and/or alkyl-modified silicone compounds, which can either be liquid or in resin form at room temperature. Also suitable are simethicones, which are mixtures of dimethicones having an average chain length of from 200 to 300 dimethylsiloxane units and hydrogenated silicates. A detailed review of suitable volatile silicones can additionally be found in Todd et al., Cosm. Toil. 91, 27 (1976).

### UV Light Protection Filters and Antioxidants

UV light protection factors are, for example, to be understood as meaning organic substances (light protection filters) which are liquid or crystalline at room temperature and which are able to absorb ultra-violet rays and give off the absorbed energy again in the form of longer-wavelength radiation, e.g. heat. UVB filters can be oil-soluble or water-soluble. Examples of oil-soluble substances are:

3-benzylidenecamphor or 3-benzylidenenorcamphor and derivatives thereof, e.g. 3-(4-methylbenzylidene)-camphor, as described in EP 0693471 B1;

4-aminobenzoic acid derivatives, preferably 2-ethylhexyl 4-(dimethylamino)benzoate, 2-octyl 4-(dimethylamino)benzoate and amyl 4-(dimethylamino)-benzoate;

esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methyl-benzophenone, 2,2′-dihydroxy-4-methoxybenzophenone;

esters of benzalmalonic acid, preferably di-2-ethylhexyl 4-methoxybenzmalonate;

triazine derivatives, such as, for example, 2,4,6-trianilino(p-carbo-2′-ethyl-1′-hexyloxy)-1,3,5-triazine and octyltriazone, as described in EP 0818450 A1 or dioctylbutamidotriazone (Uvasorb® HEB);

propane-1,3-diones, such as, for example, 1-(4-tert-butylphenyl)-3-(4′-methoxyphenyl)propane-1,3-dione;

ketotricyclo(5.2.1.0)decane derivatives, as described in EP 0694521 B1.

Suitable Water-soluble Substances are:

2-phenylbenzimidazole-5-sulfonic acid and the alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)-benzenesulfonic acid and 2-methyl-5-(2-oxo-3-bornylidene)sulfonic acid and salts thereof.

Suitable typical UV-A filters are, in particular, derivatives of benzoylmethane, such as, for example, 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF). The UV-A and UV-B filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Advantageously, such combinations are combined with water-soluble filters such as, for example, 2-phenylbenzimidazole-5-sulfonic acid and their alkali metal, alkaline earth metal, ammonium, alkylammonium, alkanolammonium and glucammonium salts.

As well as said soluble substances, insoluble light protection pigments, namely finely dispersed metal oxides or salts, are also suitable for this purpose. Examples. of suitable metal oxides are, in particular, zinc oxide and titanium dioxide and also oxides of iron, zirconium, silicon, manganese, aluminum and cerium, and mixtures thereof. Salts which may be used are silicates (talc), barium sulfate or zinc stearate. The oxides and salts are used in the form of the pigments for skincare and skin-protective emulsions and decorative cosmetics. The particles here should have an average diameter of less than 100 nm, preferably between 5 and 50 nm and in particular between 15 and 30 nm. They can have a spherical shape, but it is also possible to use particles which have an ellipsoidal shape or a shape deviating in some other way from the spherical form. The pigments can also be surface-treated, i.e. hydrophilicized or hydrophobicized. Typical examples are coated titanium dioxides, such as, for example, titanium dioxide T 805 (Degussa) or Eusolex® T2000 (Merck). Suitable hydrophobic coating agents are here primarily silicones and, specifically in this case, trialkoxyoctylsilanes or simethicones. In sunscreens, preference is given to using micro- or nanopigments. Preference is given to using micronized zinc oxide. Further suitable UV light protection filters are given in the review by P. Finkel in SÖFW-Journal 122, 543 (1996) and Parf. Kosm. 3, 11 (1999).

As well as the two abovementioned groups of primary light protection substances, it is also possible to use secondary light protection agents of the antioxidant type; these interrupt the photochemical reaction chain which is triggered when UV radiation penetrates the skin. Typical examples thereof are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of gum benzoin, rutic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄) selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

### Biogenic Active Ingredients

Biogenic active ingredients are understood as meaning, for example, tocopherol, tocopherol acetate, tocopherol palmitate, ascorbic acid, (deoxy)ribonucleic acid and fragmentation products thereof, β-glucons, retinol, bisabolol, allantoin, phytantriol, panthenol, AHA acids, amino acids, ceramides, pseudoceramides, essential oils, plant extracts, such as, for example, prune extract, bambara nut extract, and vitamin complexes.

### Deodorants and Antimicrobial Agents

Cosmetic deodorants counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents.

### Antimicrobial Agents

Suitable antimicrobial agents are, in principle, all substances effective against gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorohexidine, 3,4,4′-trichlorocarbanilide (TTC), antibacterial fragrances, thymol, thyme oil, eugenol, oil of cloves, menthol, mint oil, farnesol, phenoxyethanol, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide.

Enzyme Inhibitors

Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen® CAT). The substances inhibit enzyme activity, thereby reducing the formation of odor. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Odor Absorbers

Suitable odor absorbers are substances which are able to absorb and largely retain odor-forming compounds. They lower the partial pressure of the individual components, thus also reducing their rate of diffusion. It is important that in this process perfumes must remain unimpaired. Odor absorbers are not effective against bacteria. They comprise, for example, as main constituent, a complex zinc salt of ricinoleic acid or specific, largely odor-neutral fragrances which are known to the person skilled in the art as “fixatives”, such as, for example, extracts of labdanum or styrax or certain abietic acid derivatives. The odor masking agents are fragrances or perfume oils, which, in addition to their function as odor masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethol, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Ethereal oils of relatively low volatility, which are mostly used as aroma components, are also suitable as perfume oils, e.g. sage oil, camomile oil, oil of cloves, melissa oil, mint oil, cinnamon leaf oil, linden flower oil, juniper berry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Antiperspirants

Antiperspirants reduce the formation of perspiration by influencing the activity of the eccrine sweat glands, thus counteracting underarm wetness and body odor. Aqueous or anhydrous formulations of antiperspirants typically comprise the following ingredients:

astringent active ingredients,

oil components,

nonionic emulsifiers,

coemulsifiers,

bodying agents,

auxiliaries, such as, for example, thickeners or complexing agents and/or

nonaqueous solvents, such as, for example, ethanol, propylene glycol and/or glycerol.

Suitable astringent antiperspirant active ingredients are primarily salts of aluminum, zirconium or of zinc. Such suitable antihydrotic active ingredients are, for example, aluminum chloride, aluminum chlorohydrate, aluminum dichlorohydrate, aluminum sesquichlorohydrate and complex compounds thereof, e.g. with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, e.g. with amino acids, such as glycine. In addition, customary oil-soluble and water-soluble auxiliaries may be present in antiperspirants in relatively small amounts. Such oil-soluble auxiliaries may, for example, be:

anti-inflammatory, skin-protective or perfumed ethereal oils,

synthetic skin-protective active ingredients and/or

oil-soluble perfume oils.

Customary water-soluble additives are, for example, preservatives, water-soluble fragrances, pH regulators, e.g. buffer mixtures, water-soluble thickeners, e.g. water-soluble natural or synthetic polymers, such as, for example, xanthan gum, hydroxyethylcellulose, polyvinylpyrrolidone or high molecular weight polyethylene oxides.

### Film Formers

Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof, and similar compounds.

### Swelling Agents

The swelling agents for aqueous phases may be montmorillonites, clay mineral substances, Pemulen, and alkyl-modified Carbopol grades (Goodrich). Other suitable polymers and swelling agents are given in the overview by R. Lochhead in Cosm. Toil. 108, 95 (1993).

### Insect Repellents

Suitable insect repellents are N,N-diethyl-m-toluamide, 1,2-pentanediol or ethyl butylacetylaminopropionate.

### Self-tanning Agents and Depigmentation Agents

A suitable self-tanning agent is dihydroxyacetone. Suitable tyrosine inhibitors, which prevent the formation of melanin and are used in depigmentation agents, are, for example, arbutin, ferulic acid, kojic acid, coumaric acid and ascorbic acid (vitamin C).

### Hydrotropes

To improve the flow behavior, hydrotropes, such as, for example, ethanol, isopropyl alcohol, or polyols, can also be used. Polyols which are suitable here preferably have 2 to 15 carbon atoms and at least two hydroxyl groups. The polyols can also contain further functional groups, in particular amino groups, or be modified with nitrogen. Typical examples are

glycerol;

alkylene glycols, such as, for example, ethylene glycol, diethylene glycol, propylene glycol, butylene glycol, hexylene glycol, and polyethylene glycols with an average molecular weight of from 100 to 1000 daltons;

technical-grade oligoglycerol mixtures with a degree of self-condensation of from 1.5 to 10, such as, for example, technical-grade diglycerol mixtures with a diglycerol content of from 40 to 50% by weight;

methylol compounds, such as, in particular, trimethylolethane, trimethylolpropane, trimethylolbutane, pentaerythritol and dipentaerythritol;

lower alkyl glucosides, in particular those with 1 to 8 carbon atoms in the alkyl radical, such as, for example, methyl and butyl glucoside;

sugar alcohols with 5 to 12 carbon atoms, such as, for example, sorbitol or mannitol,

sugars with 5 to 12 carbon atoms, such as, for example, glucose or sucrose;

amino sugars, such as, for example, glucamine;

dialcohol amines, such as diethanolamine or 2-amino-1,3-propanediol.

### Preservatives

Suitable preservatives are, for example, phenoxyethanol, formaldehyde solution, parabenes, pentanediol or sorbic acid, as well as the silver complexes known under the name Surfacine® and the other classes of substance listed in Annex 6, Part A and B of the Cosmetics Directive.

### Perfume Oils and Aromas

Perfume oils which may be mentioned are mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pine wood, sandalwood, guaiac wood, cedarwood, rosewood), herbs and grasses (tarragon, lemon grass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include predominantly the terpenes and balsams. Preference is, however, given to using mixtures of different fragrances which together produce a pleasing fragrance note. Ethereal oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Suitable aromas as, for example, peppermint oil, spearmint oil, anise oil, steranis oil, cumin oil, eucalpytus oil, fennel oil, lemon oil, wintergreen oil, oil of cloves, menthol and the like.

### Dyes

Dyes which can be used are the substances which are approved and suitable for cosmetic purposes, as are summarized, for example, in the publication “Kosmetische Färbemittel” [Cosmetic Colorants] from the Farbstoffkonmmission der Deutschen Forschungsgemeinschaft [Dyes Commission of the German Research Council], Verlag Chemie, Weinheim, 1984, pp. 81-106. Examples are cochineal red A (C.I. 16255), Patent Blue V (C.I. 42051), Indigo tin (C.I. 73015), Clorophyllin (C.I. 75810), quinoline yellow (C.I. 47005), titanium dioxide (C.I. 77891), Indanthrene Blue RS (C.I. 69800) and Madder Lake (C.I. 58000). Luminol may also be present as luminescent dye. These dyes are normally used in concentrations of from 0.001 to 0.1% by weight, based on the total mixture.

The total amount of auxiliaries and additives may be 1 to 50% by weight, preferably 5 to 40% by weight, based on the compositions. The compositions can be prepared by customary cold or hot processes; preference is given to working in accordance with the phase inversion temperature method.

### EXAMPLES

### A. Effectiveness Against Free Radicals

The effectiveness of the OPCs against free radicals was investigated both chemically and also biochemically by various methods:

Method A In a first method, use was made of diphenylpicrylhydrazyl (DPPH^{o}), a relatively stable free radical which produces a purple-colored solution. The parameter determined was the optical density (DO) at 513 nm.

Method B In the presence of iron(II)ions and EDTA, hydroxyl radicals were liberated from hydrogen peroxide and used for the oxidation of deoxyribose. The oxidation product forms a pink-colored compound with thiobarbituric acid. Its concentration corresponds to the optical density at 532 nm. It was investigated whether less deoxyribose is oxidized, i.e. fewer free radicals are liberated, in the presence of the test products.

Method C The experiment described above was investigated in the absence of EDTA in order to check the suitability of the test substances for forming inactive iron complexes.

Method D Xanthin oxidase is an enzyme which is released as a result of oxidative stress and catabolizes the degradation of the purine bases adenine and guanine into uronic acid and superoxide anions. The latter dismutate spontaneously or in the presence of superoxide dismutase into hydrogen peroxide and oxygen.

The amount of superoxide anion can be determined by NBT reduction via the optical density at 490 nm. It was investigated whether fewer superoxide anions are generated or more anions are destroyed in the presence of the test substances.

The results are summarized in Table 1; in each case the EC50 values are given in % (w/v).

TABLE 1
Effect against free radicals

| | | Test method | | | |
|---|---|---|---|---|---|
| Ex. | Test Product | A | B | C | D |
| 1 | Green tea | 0.0009 | At 0.03% = 15 ± 7 | At 0.03% = 54 ± 18 | 0.0009% |
| | extract | | | | |
| 2 | Pine bark | 0.0018 | At 0.03% = 10 ± 0 | At 0.03% = 47 ± 24 | 0.0053% |
| | extract | | | | |
| 3 | Grape seed | 0.0015 | At 0.03% = 5 ± 4 | At 0.03% = 49 ± 24 | 0.0049% |
| | extract | | | | |
| 4 | Litchi | 0.0010 | At 0.03% = 18 ± 3 | At 0.03% = 62 ± 6 | 0.0100% |
| | extract | | | | |
| C1 | Tocopherol | 0.0067 | n.d. | n.d. | no |
| | | | | | effect |
| C2 | Ascorbic acid | 0.0013 | 0.2607% | 0.9% | 0.5909% |
| C3 | BHT* | 0.0500 | n.d. | n.d. | no |
| | | | | | effect |

*BHT = butylhydroxytoluene

The results lead to the conclusion that the OPC-containing extracts have an antioxidative effectiveness which is significantly greater than that of tocopherol and BHT and is in the same order of magnitude as vitamin C. The OPCs have a particularly high potential for quenching hydroxyl ions in the absence of EDTA, which demonstrates that they form stable iron complexes. Finally, they have a strong potential for preventing the reduction of BT by superoxide anions.

### B. Cell Protection Against UV-A Rays

The object of the following in vitro investigations was to establish whether OPC or OPC-containing extracts are able to protect human fibroblasts against oxidative stress, specifically the effect of UV-A rays. UV-A was chosen as stress factor since the rays penetrate into the dermis where, in particular, they bring about lipoperoxidation of the cytoplasm membranes. The lipoperoxide formed degrade into malonaldialdehyde (MDAs), which are responsible for the reticulation of many biomolecules, such as, for example, proteins (enzyme inhibition) or nucleic bases (mutagenesis). To carry out the experiment, a fibroblast culture containing foetal calf serum was prepared and two days later inoculated with the test substances. Following incubation for 36 h at 37° C. and a CO₂ level of 5% by volume, the nutrient medium was replaced by an electrolyte solution and the fibroblasts were damaged using a defined amount of UVA radiation (3-15 J/cm²). When irradiation was complete, the amount of MDA formed in the supernatant solution was determined by reaction with thiobarbituric acid, and the content of proteins in the cell homogenisate was determined in accordance with the Bradford method. The results are summarized in Table 2. They are relative percentages compared with the standard. The values given are the average values of two measurement series with triple determination.

TABLE 2
Effect against UV-A rays

| | | Conc. | Released | Cellular |
|---|---|---|---|---|
| Ex. | Test product | [% by wt) | MDA | proteins |
| | Control without UVA | | 0 | 100 |
| | Control with UVA | | 100 | 101 |
| 5 | UVA + green tea extract | 0.001 | 63 | 96 |
| 6 | UVA + pine bark extract | 0.001 | 54 | 98 |
| 7 | UVA + grape seed extract | 0.001 | 47 | 93 |
| 8 | UVA + Litchi extract | 0.003 | 31 | 105 |

The results show that the OPC-containing extracts have a lasting positive influence in the fight against oxidative stress, without damaging the fibroblasts.

### C. Cell Protection Against UV-B Rays

The object of this test was to show that OPCs have an anti-inflammatory properties toward human keratinocytes. UVB was chosen as stress factor because the rays bring about cutaneous inflammation (erythaema, oedema) as a result of activating arachidonic acid-liberating enzymes, such as, for example, phospholipase A2 (PLA2). This consequently leads not only to damage of the membranes, but also to the formation of substances with an inflammatory effect, such as, for example, prostaglandins of the PGE2 type. The influence of the UV-B rays on the keratinocytes was determined in vitro via the release of cytoplasmic enzymes, such as, for example, LDH (lactate dehydrogenase), which proceeds in parallel to cell damage and the formation of PGE2. The keratinocyte cultures were prepared as already described under B). The amount of radiation this time was 50 mJ/cm². The amount of keratinocytes was ascertained after trypsination by means of a cell counter, which determines the LDH concentration enzymatically. The results are summarized in Table 3. The values given are the activities in relative percentages compared with a standard as an average value of two test series with duplicate determination.

TABLE 3
Action against UV-B rays

| | | Conc. | | | |
|---|---|---|---|---|---|
| | | [% by | Cellular | Released | Released |
| Ex. | Test product | wt) | DNA | LDH | PGE2 |
| | Control without UVB | | 100 | 0 | 0 |
| | Control with UVB | | 31 | 100 | 100 |
| 9 | UVB + green tea | 0.0002 | 32 | 87 | — |
| | extract | | | | |
| 10 | UVB + pine bark | 0.001 | 37 | 69 | 77 |
| | extract | | | | |
| 11 | UVB + grape seed | 0.0003 | 39 | 68 | 94 |
| | extract | | | | |
| 12 | UVB + Litchi | 0.001 | 36 | 64 | — |
| | extract | | | | |

The results show that the OPC-rich extracts significantly reduce the harmful effects of UV-B rays and, in particular, reduce the release of LDH and PGE2.

### D. Antiprotease Effectiveness

During inflammation, skin proteases, such as, for example, collagenase, are released from the polymorphonuclear neutrophilic granulocytes or macrophages. A similar process occurs particularly in the skin of older people under the influence of UV rays. The proteases—also referred to as matrix-metalloproteases (MMP) due to their content of central zinc ions—catalyze, as already mentioned, the fragmentation of connective tissue proteins. To investigate the test substances for collagenase inhibition, use was made of bacterial collagenase (clostridium histolyticum) on gelatin as natural nutrient base which was labeled with fluorochrome (FITC, Calbiochem). The incubation time was 60 min at 20° C., the hydrolysis of the substrate was monitored via the fluorescence at 393 nm (excitation at 328 nm). The results are summarized in Table 4. The collagenase inhibition is given in %.

Correspondingly, the MMP inhibition was also investigated by a biochemical route. For this, the synthetic substrate used was MCA-Pro-Leu-Gly-Leu-Dpa-Ala-Arg-NH2. For the Litchi extract, a MMP inhibition of from 13% (concentration 0.005% by weight) to 95% (0.1% by weight) was established.

TABLE 4a
Collagenase inhibition - Data in % inhibition

| | | Concentration % (w/v) | | |
|---|---|---|---|---|
| Ex. | Test product | 0.1 | 0.03 | 0.01 |
| 13 | Green tea extract | 100 | 76 ± 7 | 47 |
| 14 | Pine bark extract | 90 | 50 ± 1 | 18 |
| 15 | Grape seed extract | 100 | 55 ± 2 | 15 ± 2 |
| 16 | Litchi extract | 89 ± 4 | 53 ± 2 | 16 ± 0 |

TABLE 4b
Collagenase inhibition by A2 dimer and cysteine

| Test product | % Inhibition |
|---|---|
| A2 Dimer 0.003% | 16 |
| A2 Dimer 0.003% | 30 |
| A2 Dimer 0.003% | 71 |
| Cysteine (Sigma) 0.056% | 14 |
| Cysteine (Sigma) 0.056% | 37 |
| Cysteine (Sigma) 0.056% | 54 |
| Cysteine (Sigma) 0.056% | 84 |

The results show that the OPC-rich extracts (Table 4a) and the active substances (Table 4b) have a significant inhibiting effect which is dependent on the concentration.

### E. Inhibition of Human MMP-1 Synthesis

The ability of Litchi extract to reduce the toxic effect of UV-A rays was investigated. The in vitro system used was a culture of dermal fibroblasts, and the parameter determined was the release of MMP-1 from these fibroblasts under the influence of UV radiation. Preparation of the nutrient solution, cultivation and damage were carried out analogously to B). The MMP was determined using a kit which is available commercially under the name RPN2610 from Amersham. The results are summarized in Table 5. The values given are the amounts of MMP in ng/ml from a test series with triplicate determination.

TABLE 5
MMP Inhibition

| | |
|---|---|
| Conc. | MMP |

| Ex. | Test Product | % by wt | without UVA | with UVA |
|---|---|---|---|---|
| | Control | | 49 + 9 | 199 + 25 |
| 17 | Litchi extract | 0.0006 | 60 + 20 | 140 + 17 |
| 18 | Litchi extract | 0.003 | 82 + 11 | 164 + 8 |

The results show that OPC-rich extracts reduce the release of MMP upon UV-A irradiation with lasting effect.

### F. Anti-inflammatory Effect

In the course of cutaneous inflammation, leukocytes, such as, for example, the polymorphonuclear neutrophilic granulocytes (PMN), are stimulated by peptides, such as, for example, cytokines, to emit messenger substances, such as, for example, leucotriene, which are released from activated or necrotic cells in the dermis. These activated PMNs release not only proinflammatory cytokins, leucotrienes and proteases, but also ROS, such as, for example, superoxides and hypochlorite anions, which have the task of destroying pathogenic microbes or fungi which have penetrated it. This activity of the PMNs during inflammation is known as respiratory burst and can lead to additional tissue damage. To investigate to what extent OPCs and OPC-rich extracts are able to prevent or reduce respiratory burst, a cell line of human leucemic granulocytes of these PMNS was incubated together with the test substances at 37° C. from 5% by volume of CO₂. After the respiratory burst had been triggered by adding a yeast extract (zymosan) to the cell solution, the release of superoxide anions was determined by means of their reaction with Luminol. The results are summarized in Table 6. The values given are the cell numbers and the amount of released ROS in relative percentages compared with the standard as an average value of a measurement series with triplicate determination.

TABLE 6
Anti-inflammatory effect

| | | Conc. % | Cell | Released |
|---|---|---|---|---|
| Ex. | Test Product | by wt. | numbers | ROS |
| | Control | | 100 | 100 |
| 19 | Potentilla | 0.001 | 104 ± 1 | 37 ± 25 |
| | extract | | | |
| 20 | Potentilla | 0.01 | 94 ± 3 | 9 ± 4 |
| | extract | | | |
| 21 | Potentilla | 0.1 | 93 ± 1 | 8 ± 4 |
| | extract | | | |

The results show that the OPCs have a strong inhibiting effect on the respiratory burst of human granulocytes without damaging the granulocytes. Similar results could also be demonstrated for Litchi pericarp extract.

Table 7 below gives a number of formulation examples.

TABLE 7
Examples of cosmetic preparation (water, preservative ad
100% by weight) - contd.

| Composition (INCI) | A | B | C | D | E |
|---|---|---|---|---|---|
| Emulgade ® SE | 5.0 | 5.0 | 4.0 | — | — |
| Glyceryl Stearate (and) Ceteareth | | | | | |
| 17/20 (and) Cetearyl Alcohol (and) | | | | | |
| Cetyl Palmitate | | | | | |
| Eumulgin ® B1 | — | — | 1.0 | — | — |
| Ceteareth-12 | | | | | |
| Lameform ® TGI | — | — | — | 4.0 | — |
| Polyglyceryl-3 Isostearate | | | | | |
| Dehymuls ® PGPH | — | — | — | — | 4.0 |
| Polyglyceryl-2 Dipolyhydroxystearate | | | | | |
| Monomuls ® 90-O 18 | — | — | — | 2.0 | — |
| Glyceryl Oleate | | | | | |
| Cetiol ® HE | — | — | — | — | 2.0 |
| PEG-7 Glyceryl Cocoate | | | | | |
| Cetiol ® OE | — | — | — | 5.0 | 6.0 |
| Dicaprylyl Ether | | | | | |
| Cetiol ® PGL | — | — | 3.0 | 10.0 | 9.0 |
| Hexyldecanol (and) Hexyldecyl Laurate | | | | | |
| Cetiol ® SN | 3.0 | 3.0 | — | — | — |
| Cetearyl Isononanoate | | | | | |
| Cetiol ® V | 3.0 | 3.0 | — | — | — |
| Decyl Oleate | | | | | |
| Myritol ® 318 | — | — | 3.0 | 5.0 | 5.0 |
| Coco Caprylate Caprate | | | | | |
| Beeswax | — | — | — | 7.0 | 5.0 |
| Nutrilan ® Elastin E20 | 2.0 | — | — | — | — |
| Hydrolyzed Elastin | | | | | |
| Nutrilan ® I-50 | — | 2.0 | — | — | — |
| Hydrolyzed Collagen | | | | | |
| Gluadin ® AGP | — | — | 0.5 | — | — |
| Hydrolyzed Wheat Gluten | | | | | |
| Gluadin ® WK | — | — | — | 0.5 | 0.5 |
| Sodium Cocoyl Hydrolyzed Wheat | | | | | |
| Protein | | | | | |
| Pine Bark Extract | 1.0 | 1.0 | 1.0 | 1.0 | 1.0 |
| Grape Seed Extract | 1.0 | 1.0 | 1.0 | 1.0 | 1.0 |
| Hydagen ® CMF | 1.0 | 1.0 | 1.0 | 1.0 | 1.0 |
| Chitosan | | | | | |
| Magnesium Sulfate Hepta Hydrate | — | — | — | 1.0 | 1.0 |
| Glycerol (86% by weight) | 3.0 | 3.0 | 5.0 | 5.0 | 3.0 |

(A) soft cream, (B, C) moisturizing emulsion, (D, E) night cream

1-14. (Canceled)

15. A method of treating skin to counteract the effects of ageing, said method comprising:
(a) providing a skin substrate to be treated; and
(b) contacting the skin substrate with a cosmetic preparation comprising at least one oligomeric proanthocyanidin.

16. The method according to claim 15, wherein the at least one oligomeric proanthocyanidin comprises an A2 dimer.

17. The method according to claim 15, wherein the at least one oligomeric proanthocyanidin comprises A2 proanthocyanidin.

18. The method according to claim 15, wherein the at least one oligomeric proanthocyanidin is present as a constituent of a plant extract.

19. The method according to claim 18, wherein the plant extract is selected from the group consisting of extracts of green tea, pine bark, grape seed, Litchi pericarp, Potentilla and mixtures thereof.

20. A method of reducing the activity and the output of matrix-metallo proteases in fibroblasts, said method comprising:
(a) providing a dermal region having one or more fibroblasts to be treated; and
(b) contacting the dermal region with a cosmetic preparation comprising at least one oligomeric proanthocyanidin.

21. The method according to claim 20, wherein the at least one oligomeric proanthocyanidin comprises an A2 dimer.

22. The method according to claim 20, wherein the at least one oligomeric proanthocyanidin comprises A2 proanthocyanidin.

23. The method according to claim 20, wherein the at least one oligomeric proanthocyanidin is present as a constituent of a plant extract.

24. The method according to claim 23, wherein the plant extract is selected from the group consisting of extracts of green tea, pine bark, grape seed, Litchi pericarp, Potentilla and mixtures thereof.

25. A method of treating a skin condition selected from the group consisting of acne, rosacea and cellulite, said method comprising:
(a) providing a skin substrate affected by one or more conditions selected from the group consisting of acne, rosacea and cellulite; and
(b) contacting the skin substrate with a cosmetic preparation comprising at least one oligomeric proanthocyanidin.

26. The method according to claim 25, wherein the at least one oligomeric proanthocyanidin comprises an A2 dimer.

27. The method according to claim 25, wherein the at least one oligomeric proanthocyanidin comprises A2 proanthocyanidin.

28. The method according to claim 25, wherein the at least one oligomeric proanthocyanidin is present as a constituent of a plant extract.

29. The method according to claim 28, wherein the plant extract is selected from the group consisting of extracts of green tea, pine bark, grape seed, Litchi pericarp, Potentilla and mixtures thereof.

30. A method of forming inactive iron cation complexes and deactivating superoxide anions, said method comprising:
(a) providing a skin substrate containing ionic constituents selected from the group consisting of liberated iron cations and superoxide anions; and
(b) contacting the skin substrate with a cosmetic preparation comprising at least one oligomeric proanthocyanidin.

31. The method according to claim 30, wherein the at least one oligomeric proanthocyanidin comprises an A2 dimer.

32. The method according to claim 30, wherein the at least one oligomeric proanthocyanidin comprises A2 proanthocyanidin.

33. The method according to claim 30, wherein the at least one oligomeric proanthocyanidin is present as a constituent of a plant extract.

34. The method according to claim 32, wherein the plant extract is selected from the group consisting of extracts of green tea, pine bark, grape seed, Litchi pericarp, Potentilla and mixtures thereof.
